(19) United States
(12) Patent Application Publication
(10) Pub. No.: US 2009/0326372 A1
(43) Pub. Date: Dec. 31, 2009

(54) Compound Imaging with HIFU Transducer and Use of Pseudo 3D Imaging

(76) Inventors: Gregory DARLINGTON, Snohomish, WA (US); Michael LAU, Edmonds, WA (US); Michael CONNOLLY, Bothell, WA (US)

Correspondence Address: SHAY GLENN LLP, 2755 CAMPUS DRIVE, SUITE 210, SAN MATEO, CA, 94403, US

(21) Appl. No.: 12/165,346

(22) Filed: Jun. 30, 2008

(51) Int. Cl. A61N 7/00; A61B 8/00

(52) U.S. Cl. 600/439

(57) ABSTRACT

A medical imaging and therapy applicator is provided that may include any of a number of features. One feature of the medical imaging and therapy applicator is that it can image target tissue of a patient with ultrasound imaging transducers. Another feature of the medical imaging and therapy applicator is that it can display the imaging information and provide therapeutic energy to the target tissue. Methods associated with use of the medical imaging and therapy applicator are also covered.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present application is direct to methods and apparatus that provide ultrasound imaging and therapeutic treatment of internal pathological conditions using high intensity focused ultrasound energy.

### BACKGROUND OF THE INVENTION

Ultrasound is used in medical settings as a diagnostic aid for imaging internal structures. Advantages of ultrasound over other imaging forms include low cost, portability, and safety.

A variety of therapeutic uses of ultrasound have been described. Some applications take advantage of the ability of high intensity focused ultrasound (HIFU) waves to generate heat and thus destroy tissue such as tumors or blood vessels. Currently, HIFU is being used for the treatment of uterine fibroid tumors, prostate hyperplasia or cancer, liver cancer, malignant bone and soft tissue sarcoma, and internal bleeding.

By focusing HIFU energy at a desired delivery site such as, e.g., a tumor, higher local concentrations of a therapeutic agent may be achieved. HIFU treatment can utilize thermal and mechanical mechanisms to treat target tissue. The focal temperature at the HIFU focal point can quickly exceed 70° C., and thereafter reach 100° C. depending on the application of HIFU energy. However, when the HIFU energy is focused at locations deep in tissue, the intervening tissue between the HIFU source and the HIFU focal point remains unharmed.

In applications where HIFU energy is delivered to internal body tissue, it is preferred that the target tissue along with the surrounding tissues be visualized in real time throughout the HIFU treatment. Presently, MRI is being used to guide HIFU treatment of internal fibroids. Transabdominal ultrasound-guided HIFU treatment of liver tumors and uterine fibroids is also being practiced. These approaches require bulky and bandwidth intensive imaging equipment to be used external to the tissue to be treated.

Accordingly, the present invention is directed to provide consistent low processing-bandwidth clear imaging of target tissue and the HIFU effect at the target tissue to help guide movement of the HIFU focal point throughout the procedure.

### SUMMARY OF THE INVENTION

Generally, the present invention contemplates the use of ultrasound imaging and therapeutic treatment of internal pathological conditions using high intensity focused ultrasound energy.

One aspect of the invention provides a medical imaging and therapy applicator having an applicator body and a pair of ultrasound imaging transducers disposed on the applicator body. The pair of ultrasound imaging transducers can be adapted to provide a two-dimensional image along one image plane. The applicator can be adapted to be inserted into a cavity of a patient for the treatment of target tissue within or not within the cavity. The applicator can also be used in non-endocavity applications. In one embodiment, multiple pairs of ultrasound imaging transducers provide multiple two-dimensional imaging planes. In the preferred embodiment, the therapy transducer is a HIFU transducer.

Another aspect of the invention provides a display for visualizing the target volume of a medical imaging and therapy applicator. In one embodiment, the display provides a two-dimensional image of the target location. In another embodiment, the display simultaneously provides two-dimensional images of the target location along more than one imaging plane. In yet another embodiment, a user can select which sections of the imaging planes are to be displayed on the display.

One aspect of the invention provides a method for imaging and treating a patient with therapeutic ultrasound. The method includes the steps of inserting an applicator into the cavity of a patient; imaging the target tissue with the ultrasound imaging transducers of the applicator; and providing therapy to the target tissue with the therapy transducer of the applicator.

Another aspect of the invention provides a method for treating a target tissue of a patient with therapeutic energy. The method includes the steps of generating a two-dimensional ultrasound image of the target tissue along one image plane with a pair of ultrasound imaging transducers disposed on an applicator body; and providing therapeutic energy from a therapy transducer disposed on the applicator body.

One aspect of the invention provides a medical imaging and therapy applicator having an applicator body and a plurality of transducers disposed on the applicator body. Pairs of transducers can be distance separated and combine to form two-dimensional images of a target location. The transducers can also apply therapeutic energy to the target location.

Yet another aspect of the invention provides a medical imaging and therapy system having an applicator body, a plurality of ultrasound imaging transducers disposed on the body, a therapy transducer disposed on the body, a display configured to display images generated by the imaging transducers in at least two image planes, and an electronic mechanism configured to rotate the at least two image planes.

One aspect of the invention provides a method of treating a patient with therapeutic ultrasound. The method includes the steps of imaging target tissue with an applicator that supports at least one pair of distance-separated imaging transducers that combine to provide two-dimensional images, and providing therapy to the target tissue with a therapy transducer supported by the applicator.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which.

FIG. 1 illustrates a medical imaging and therapy applicator according to one embodiment of the present invention;

FIGS. 2A-2D illustrate various configurations of imaging transducers on a therapy applicator according to some embodiments of the present invention;

FIG. 3 illustrates a medical imaging and therapy applicator having an imaging field of view intersecting with a HIFU focal point according to one embodiment of the present invention;

FIGS. 4A-4D illustrate various bi-plane display modes for visualizing the target location of a medical imaging and therapy applicator according to one embodiment of the present invention.

FIG. 5A is a schematic drawing showing the image shadow cast by an obstacle using spaced-apart imaging transducers.

FIG. 5B is a schematic drawing showing the image shadow cast by an obstacle using a single imaging transducer.

### DETAILED DESCRIPTION OF THE INVENTION

Certain specific details are set forth in the following description and figures to provide an understanding of various embodiments of the invention. Certain well-known details, associated electronics and devices are not set forth in the following disclosure to avoid unnecessarily obscuring the various embodiments of the invention. Further, those of ordinary skill in the relevant art will understand that they can practice other embodiments of the invention without one or more of the details described below. Finally, while various processes are described with reference to steps and sequences in the following disclosure, the description is for providing a clear implementation of particular embodiments of the invention, and the steps and sequences of steps should not be taken as required to practice this invention.

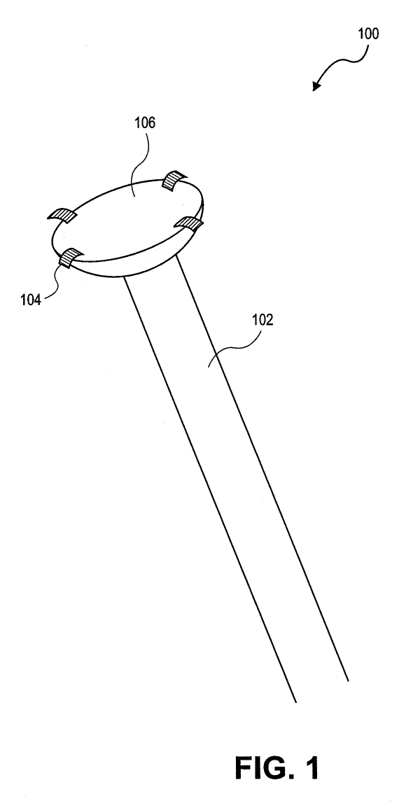

FIG. 1 illustrates a medical imaging and therapy applicator 100 comprising applicator body 102, ultrasound imaging transducers 104, and therapy transducer 106. Any of transducers 104 and/or transducer 106 may be formed as an array or other combination of elements, depending on the nature of the transducer. Applicator 100 can be adapted to be inserted into a patient cavity (not shown in FIG. 1), for the treatment of a target tissue, such as uterine fibroid tumors, prostate hyperplasia or cancer, liver cancer, malignant bone and soft tissue sarcoma, and internal bleeding, for example. In another embodiment, applicator 100 can be a non-endocavity applicator for through-skin applications such as treatment of benign breast tumors, uterine fibroids, thyroid nodules, and deep skin lesions, for example. In the preferred embodiment, therapy transducer 106 can be a high intensity focused ultrasound (HIFU) transducer. However, in other embodiments therapy transducer 106 can be a variety of transducers adapted to provide therapeutic energy to a target tissue, including, but not limited to a RF transducer, a cryogenic transducer, a morcellator (or other mechanical transducer) or a microwave needle, for example. Furthermore, therapy transducer 106 can be substantially round and have a convex shape to focus therapeutic energy at a target location. Alternatively, therapy transducer can be oval-shaped. As shown in FIG. 1, therapy transducer 106 is situated on a plane perpendicular to the applicator axis. In other embodiments, however, therapy transducer 106 can be articulatable or positioned at an angle relative to the applicator axis. Applicator 100 can utilize a fluid within the patient cavity to fill any space of non-contact between applicator 100 and the target tissue, which provides optimal transmission of ultrasound energy for both imaging and therapy. As a result, applicator 100 may include a cuff (not pictured) to help retain the fluid in the patient cavity. Further details about using a fluid can be found in U.S. application Ser. No. 11/831,048, titled “METHODS AND APPARATUS FOR ENGAGEMENT AND COUPLING OF AN INTRACAVITORY IMAGING AND HIGH INTENSITY FOCUSED ULTRASOUND PROBE,” filed Jul. 31, 2007.

Ultrasound imaging transducers 104 can be situated about therapy transducer 106 in a number of ways to achieve the desired imaging from applicator 100. In the embodiment shown in FIG. 1, ultrasound imaging transducers 104 are situated around the perimeter of therapy transducer 106 on opposite ends of diameters of therapy transducer 106. In other embodiments, imaging transducers 104 may be placed in other positions on therapy transducer 106, such as within the therapy transducer perimeter. As shown in FIG. 1, the pairs of ultrasound imaging transducers 104 are distance-separated on opposite ends of therapy transducer 106 and combine to provide two-dimensional ultrasound images along two image planes. More specifically, each pair of ultrasound imaging transducers is adapted to provide a two-dimensional ultrasound image along one image plane. Splitting the imaging transducers to form a single image from two sides can give better imaging resolution and increase the field of view. In the embodiment of FIG. 1, the applicator comprises two pairs of ultrasound imaging transducers 104 to provide two two-dimensional image planes in an orthogonal configuration. However, in other embodiments, the applicator can provide a single image plane, or can provide multiple image planes that intersect at a variety of angles.

In another embodiment, transducers disposed on the applicator body can be adapted to provide two-dimensional images of a target location and apply therapeutic energy to the target location. In this embodiment, the imaging and therapy transducers are not separate transducers, as described above, but rather, each transducer is configured to provide both imaging and therapy to the target location. Such dual-mode transducers may be linear or two-dimensional arrays and are well-known in the art, such as described in U.S. Pat. Nos. 5,823,962; 6,537,224; and 6,719,694. In one embodiment, pairs of transducers are disposed on an applicator body and are distance-separated and combine to form two-dimensional images of a target location. The pairs of transducers also apply therapeutic energy to the target location. In this embodiment, the therapeutic energy focal point may coincide with the line where the imaging planes intersect, so that a user can easily and continuously visualize the relationship between the target location and surrounding anatomy.

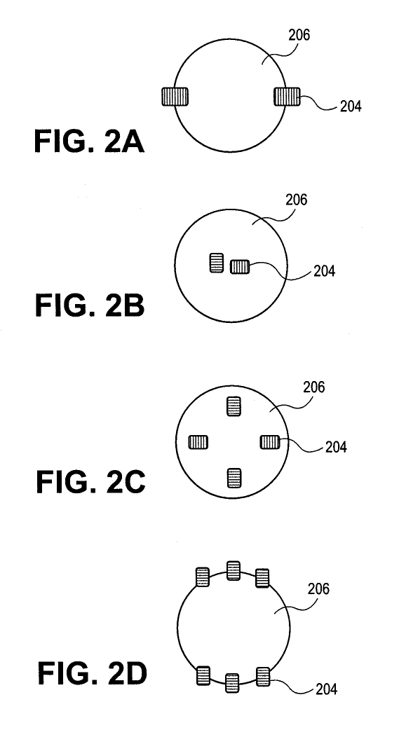

In addition to the configuration shown in FIG. 1, ultrasound imaging transducers 104 can be also be arranged in, but not limited to, the configurations illustrated in FIGS. 2A-2D. For example, FIG. 2A illustrates applicator 200 having a single pair of ultrasound imaging transducers 204, which together can provide two-dimensional ultrasound images along one image plane. FIG. 2B illustrates applicator 200 having two orthogonal imaging transducer arrays of ultrasound imaging transducers 204 situated within the perimeter of therapy transducer 206 towards the center of the therapy transducer. In contrast to the imaging transducers of FIG. 2A, the imaging arrays of FIG. 2B are not formed from distance-separated ultrasound imaging transducers, but rather, each transducer in FIG. 2B forms a single imaging transducer array. The imaging transducer arrays shown in FIG. 2B provide two-dimensional images in two image planes, the image planes being orthogonal to one another. FIG. 2C illustrates applicator 200 having two pairs of ultrasound imaging transducers 204 situated within the perimeter of therapy transducer 206. Alternatively, FIG. 2D illustrates applicator 200 having three pairs of imaging transducers 204 in a parallel configuration, to provide another method of visualizing volume of the target location. Multiple imaging transducers or transducer arrays can also be arranged in a diverging configuration, for example. In other embodiments (not shown), applicator 200 can include multiple imaging transducers 204 comprising more than two independent imaging transducer arrays adapted to provide two-dimensional ultrasound images along more than one image plane. In some embodiments, some or all of the therapy transducer may lie between the imaging transducers. For example, as can be seen in the embodiments shown in FIGS. 2A-2D, a portion of therapy transducer 206 is disposed between ultrasound imaging transducers 204.

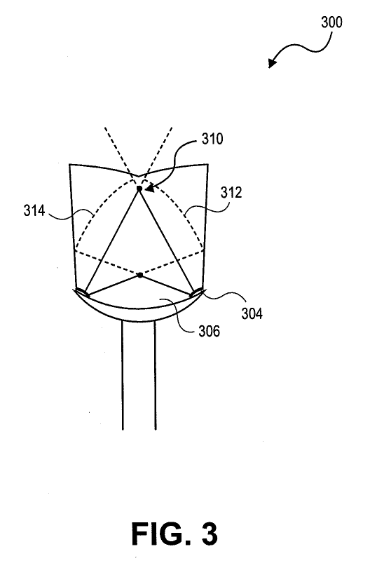

FIG. 3 illustrates medical imaging and therapy applicator 300 having an imaging field of view intersecting with a therapy focal point 310. As described above, therapy transducer 306 can be a HIFU transducer, a RF transducer, a cryogenic transducer, a microwave needle, or another appropriate type of therapy transducer, as known in the art. Two orthogonal sets of imaging transducers 304 on the distal end of applicator 300 provide two-dimensional ultrasound images along image planes 312 and 314. Therapy focal point 310 lies along the intersection of image planes 312 and 314, such that imaging transducers 304 and therapy transducer 306 are adapted to be focused at a single target location. Thus, an axis of therapy transducer 306 coincides with image planes 312 and 314. In one embodiment, it is possible to vary the location of therapy focal point 310 (e.g., therapy focal point might be adjusted axially and/or laterally) and as such, image planes 312 and 314 can also be adjusted so the imaging transducers and therapy transducer remain focused to overlap at a target location. By providing therapy focal point 310 along the intersection of image planes 312 and 314, applicator 300 can direct imaging energy and therapeutic energy or action in the same direction towards a target location. Since the therapy overlaps with the imaging, a user of the applicator will know that therapy treatment to a target location may be ineffective when obstacles (e.g. cysts, gas bubbles, fascia layers, etc.) between the applicator and the target location cause shadowing or otherwise prevent imaging of the target location.

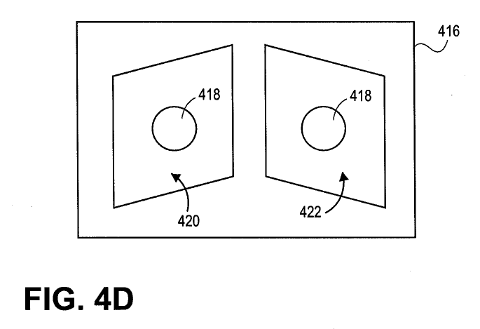

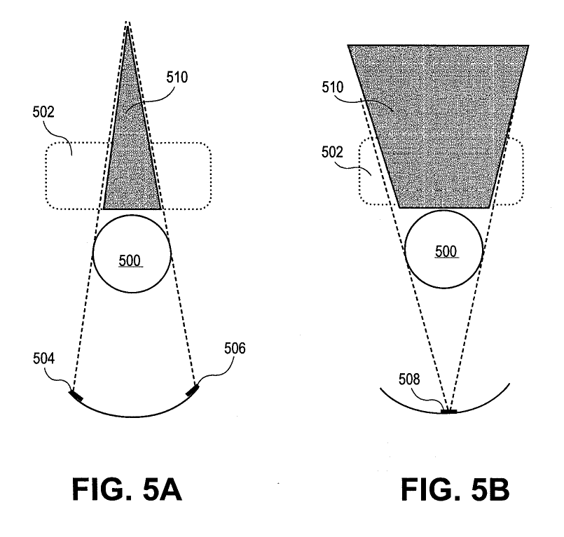

In instances in which an obstacle 500 impedes imaging of a target location 502 or impedes imaging of tissue beyond the target location, the imaging transducers 504 and 506 may be used to image around the obstacle 500 by ensuring that the spacing of the separated imaging transducers and the distance between the imaging transducers and the obstacle is such that at least a portion of the tissue behind the obstacle can be imaged by providing sufficient parallax, as shown schematically in FIG. 5A. As shown in FIG. 5B, imaging the same target region 502 with a single imaging transducer 508 results in a larger image “shadow” 510.

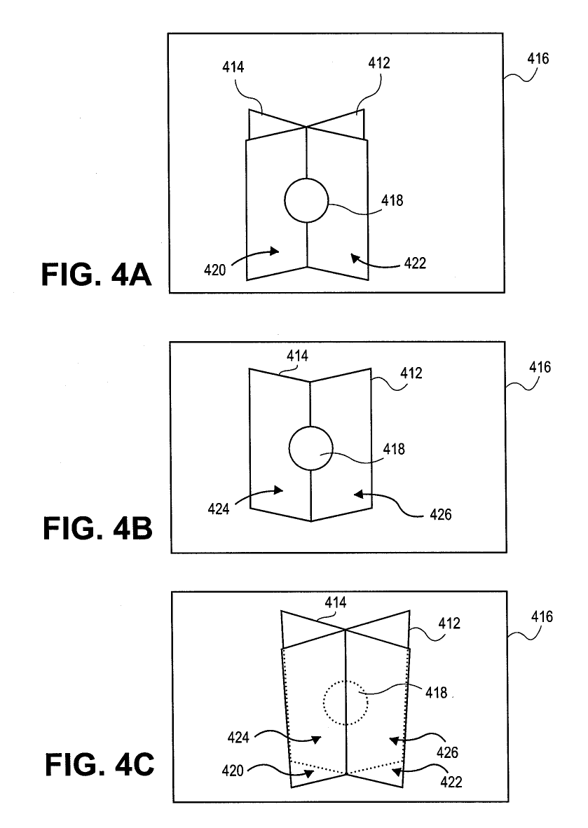

FIGS. 4A-4D illustrate various display modes for visualizing the target location of a medical imaging and therapy applicator on a two-dimensional display 416 according to some embodiments of the present invention. As described above, the applicator of FIGS. 1-3 can provide two-dimensional ultrasound images along a single imaging plane or along multiple imaging planes. As described above, the applicator can be inserted into a patient cavity or placed on the skin of a patient and provide ultrasound images of a target location within or upon the patient. When inserted into a cavity, the applicator can also provide ultrasound images of target tissue that is not located within the same patient cavity as the applicator. For example, with the applicator inserted in a patient's vagina, the imaging transducers can provide images of fibroid tumors inside the uterus, and the therapy transducer can provide HIFU to ablate the fibroid tumors inside the uterus. Display 416 can simultaneously display multiple two-dimensional images generated along the image planes of the applicator (i.e., image planes 412 and 414). Display 416 can be a CRT display, an LCD display, goggles, stereo goggles, a heads-up display, etc.

In the embodiments shown in FIGS. 4A-4D, image planes 412 and 414 are orthogonal, however, the imaging transducer arrays on the applicator need not be orthogonal in other embodiments. The manner in which image planes 412 and 414 are displayed on display 416 can be changed by a user depending on the desired view of target tissue 418. For example, in FIG. 4A, front quarter planes 420 and 422 of image planes 412 and 414, respectively, are shown on display 416. Alternatively, in FIG. 4B, rear quarter planes 424 and 426 of image planes 412 and 414, respectively, are shown on display 416. Additionally, as shown in FIG. 4C, front quarter planes 420 and 422 can be displayed in a partially transparent manner to allow visualization of front quarter planes 420 and 422 simultaneously with rear quarter planes 424 and 426. In FIG. 4D, image planes 420 and 422 are displayed separately. In embodiments where the applicator includes more than two imaging planes, display 416 can be utilized in a similar manner to simultaneously display the various front and rear quarter planes provided by the imaging arrays.

A user of the medical imaging and therapy applicator can choose how to display the imaging of the applicator. For example, the user can actively switch between display modes, such as those described in FIGS. 4A-4D, to display the desired imaging planes or portion thereof. Additionally, the user can choose to display only one image plane at a time (i.e., display only one of image planes 412 or 414), or choose to display more than two image planes simultaneously (i.e., in embodiments having more than two imaging transducer arrays). Because the applicator provides images in only two-dimensions, the amount of processing bandwidth required to process and display the ultrasound images is kept to a minimum relative to full three-dimensional imaging, which reduces the imaging system cost, size and complexity, and allows more system processing bandwidth for therapy. Additionally, since the imaging planes can be displayed simultaneously on the display, the user can easily visualize a three-dimensional image. This visualization can be aided by a slight manual rotation or movement of the applicator to verify the boundaries or shape of the target tissue. In one embodiment, the applicator includes a mechanism configured to mechanically rotate the portion of the applicator on which the imaging arrays lie in order to cause rotation of the two-dimensional imaging planes, so as to aid visualization. In this embodiment, the rotation may occur in response to user input, or may be configured for continuous rotation back and forth through angles and at speeds input by the user. In the case of a two-dimensional imaging array, rotation of the displayed image planes may be accomplished electronically (i.e. a commonly-understood feature of such 2D arrays is the ability to arbitrarily select the image plane[s] one wants to view).

One embodiment of the invention uses a HIFU therapy transducer along with ultrasound imaging transducers. In order to display ultrasound images simultaneously with HIFU treatment, one or more of the approaches described in U.S. Pat. Appln. No. 2006/0264748 and U.S. Pat. No. 6,425,867 may be employed so as to eliminate interference in the displayed image.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A medical imaging and therapy applicator comprising:
an applicator body;
a therapy transducer disposed on the applicator body; and
a pair of ultrasound imaging transducers disposed on the applicator body, wherein the pair of ultrasound imaging transducers is adapted to provide a two-dimensional image along one image plane.

2. The applicator of claim 1 further comprising a second pair of ultrasound imaging transducers adapted to provide a two-dimensional image along a second image plane.

3. The applicator of claim 2 wherein the two image planes are orthogonal.

4. The applicator of claim 2 wherein the two image planes are intersecting.

5. The applicator of claim 1 wherein at least a portion of the therapy transducer is disposed between ultrasound imaging transducers.

6. The applicator of claim 5 wherein the ultrasound imaging transducers are disposed on a periphery of the therapy transducer.

7. The applicator of claim 5 wherein the ultrasound imaging transducers are disposed within a periphery of the therapy transducer.

8. The applicator of claim 5 wherein the therapy transducer is substantially round, the imaging transducers being on opposite ends of a diameter of the therapy transducer.

9. The applicator of claim 1 wherein an axis of the therapy transducer coincides with an image plane of the ultrasound imaging transducers.

10. The applicator of claim 1 wherein the imaging transducers and the therapy transducer are disposed to direct imaging energy and therapeutic energy or action in the same direction.

11. The applicator of claim 1 wherein the applicator body is adapted to be inserted into a patient cavity.

12. The applicator of claim 1 wherein the therapy transducer is a HIFU transducer.

13. The applicator of claim 1 wherein the therapy transducer is a RF transducer.

14. The applicator of claim 1 wherein the therapy transducer is a cryogenic transducer.

15. The applicator of claim 1 wherein the therapy transducer is a microwave needle.

16. The applicator of claim 1 further comprising a display adapted to simultaneously display at least two separate images generated by the imaging transducers in at least two image planes.

17. The applicator of claim 16 wherein the display is adapted to simultaneously display two orthogonal images generated by the imaging transducers.

18. The applicator of claim 16 wherein the therapy transducer is a HIFU transducer.

19. The applicator of claim 16 wherein the applicator body is adapted to be inserted into a patient cavity.

20. A method of treating a patient with therapeutic ultrasound comprising:
inserting an applicator into a cavity of the patient, the applicator comprising a plurality of ultrasound imaging transducers and a therapy transducer;
imaging target tissue with the imaging transducers; and
providing therapy to the target tissue with the therapy transducer.

21. A method of treating a target tissue of a patient with therapeutic energy comprising:
generating a two-dimensional ultrasound image of the target tissue along one image plane with a pair of ultrasound imaging transducers disposed on an applicator body; and
providing therapeutic energy from a therapy transducer disposed on the applicator body.

22. The method of claim 21 further comprising generating a second two-dimensional ultrasound image of the target tissue along a second image plane with a second pair of ultrasound imaging transducers disposed on the applicator body.

23. The method of claim 22 wherein the two image planes are orthogonal.

24. The method of claim 22 wherein the two image planes are intersecting.

25. The method of claim 21 wherein the step of providing therapeutic energy comprises providing high intensity focused ultrasound to the target tissue.

26. The method of claim 21 wherein the step of generating a two-dimensional ultrasound image comprises directing imaging ultrasound from the applicator in a direction from the applicator, the step of providing therapeutic energy comprising directing therapeutic energy from the applicator in the same direction.

27. The method of claim 21 wherein the step of providing therapeutic energy comprises directing therapeutic energy from a location of the applicator between the pair of ultrasound imaging transducers.

28. The method of claim 22 wherein the step of providing therapeutic energy comprises directing therapeutic energy from a location of the applicator between the pairs of ultrasound imaging transducers.

29. A medical imaging and therapy applicator comprising:
an applicator body;
a plurality of transducers disposed on the applicator body, wherein pairs of transducers are distance-separated and combine to form two-dimensional images of a target location and apply therapeutic energy to the target location.

30. A medical imaging and therapy applicator comprising:
an applicator body;
a therapy transducer disposed on the applicator body; and
a plurality of ultrasound imaging transducers disposed on the applicator body, wherein pairs of ultrasound imaging transducers are distance-separated and combine to form a two-dimensional image along one image plane.

31. A medical imaging and therapy system comprising:
an applicator comprising an applicator body, a plurality of ultrasound imaging transducers disposed on the applicator body, and a therapy transducer disposed on the applicator body;
a display adapted to simultaneously display at least two separate images generated by the imaging transducers in at least two image planes; and
an electronic mechanism configured to rotate the at least two image planes.

32. A method of treating a patient with therapeutic ultrasound comprising:
imaging target tissue with an applicator that supports at least one pair of distance-separated imaging transducers that combine to provide two-dimensional images; and
providing therapy to the target tissue with a therapy transducer supported by the applicator.
